(19) United States
(12) Patent Application Publication
(10) Pub. No.: US 2015/0292126 A1
(43) Pub. Date: Oct. 15, 2015

(54) Device and Method for Removal and Prevention of Pathogens on Touch Screens

(71) Applicant: Robert Prosser, Carlsbad, CA (US)

(72) Inventor: Robert Prosser, Carlsbad, CA (US)

(21) Appl. No.: 14/251,313

(22) Filed: Apr. 11, 2014

(51) Int. Cl. D02G 3/44; A61L 2/232; A01N 25/34; D06M 11/46; D06M 11/83; G06F 3/041; D06M 11/44

(52) U.S. Cl. CPC D02G 3/449; G06F 3/041; A61L 2/232; D06M 11/44; D06M 11/46; D06M 11/83; A01N 25/34; D06M 2200/00

(57) ABSTRACT

A pathogen removal and deterrent device and system is provided for touch screens used for user input on computing devices. Using a flexible applicator placed in a biased contact with the touch screen, metallic nanoparticles infused to the applicator eradicate pathogens and deter their return.

### BACKGROUND OF THE INVENTION

1. Field of the Invention

The present device relates to disinfecting surfaces. More particularly, the device and method herein, relate to disinfecting electronic touch screens and the like which become home to a wide variety of pathogens in a short amount of time. More specifically, the device and method employ a carrier component such as a microfiber cloth which is populated with highly ionic metal oxide particles which when placed in contact with a touch screen such as on a smartphone or pad computer, will kill germs, bacteria, and other pathogens on the surface of the touchscreen and leave an ionic residue to continue disinfection of the surface subsequently.

2. Prior Art

Surfaces which are frequently touched by humans have been found to serve as reservoirs for the spread of pathogens such as viruses, bacteria, germs, and fungi which both colonize and persist upon such surfaces. Many surfaces suffer from this problem such as buttons on ATM machines, handrails on escalators, door handles on public buildings, and other surfaces where humans frequently and sequentially touch.

Such pathogens especially thrive on surfaces such as the touch screens which are modernly employed on smartphones, laptops, and pad computers and ATM machines and the like.

Further, once occupying these surfaces, such pathogens can survive for surprisingly long periods of time, for example a month or more. Consequently, each subsequent user touching a surface has the potential to acquire pathogens deposited by others from minutes before to weeks prior to a touch of a surface such as a touch screen.

Still further, many bacteria have become resistant to conventional means for their removal. Scientists have observed recently that the cell membranes of many disease-causing bacteria develop resistance to existing antibiotics and the like, by changing their electrical charge from negative to positive. Modern antibiotics work to remove bacteria because they carry a positive charge which attracts the antibiotic component to negatively charged bacteria cells. Because of the opposite charges between the two, antibiotics can penetrate and kill bacteria.

However, according to an article by Ohio State University, these bacterial pathogens have learned to adapt by changing their naturally occurring negative charge to positive. This allows bacteria cells which have learned to establish such a protective “coat” to repel the antibiotic. Consequently, many of the bacterial pathogens deposited on touch interface surfaces are likely to be resistant to conventional antibiotic material formerly used to remove them.

Adding to the problem of charge and bacteria removal is the operation of touch screen interfaces. While there are a large number of touch screen designs, two of the most employed types of touch screens employed for interfacing with a computer using a graphic interface are resistive type screens and capacitive screens.

Resistive screens have a soft protective layer on which the physical pressure of a finger (or object) creates a change in the flow of electricity on the screen's grid. This finger contact accounts for a physical bending of the screen to position the two electrically conductive layers being deflected to touch one another. One of these two layers is resistive and the other layer of the two is conductive. When the two layers in communication with a finger or contact component are deflected and pressed together, at a point on the screen, an electrical current changes at the point of contact. Interface software running on such devices is written to discern a change in the current at the location of the pixels in the area of finger contact, or the x-y coordinates thereof. Once so recognized the software causes an order or command to be initiated to carry out the function or command which corresponds with that X-Y location discerned. Capacitive touch screens have a hard protective layer and unlike resistive touch screens, capacitive screens do not use the pressure of the user's finger to cause a change in the flow of electricity at the given point on the screen pixel grid. Instead, capacitive touch screens will cause a change in the flow of electricity when contacted with anything which holds an electrical charge, including human skin. Such charges can be either a positive charge or negative charge and cause a sensed change at a position on the screen which correlates to software monitoring the input, as a command.

Capacitive touch screens are constructed from materials such as copper or indium tin oxide which store electrical charges within an electrostatic grid of tiny wires in the screen, each smaller than a human hair. There are two main types of capacitive touch screens including “surface” and “projective” touch screens which are conventionally employed for user input based on contact with positions on the screen relating to commands or icons.

Surface capacitive screens employ a plurality of sensors at the corners and a thin evenly distributed film across the surface. Projective capacitive touch screens employ a grid formed of rows and columns of tiny wires and have a separate electronic circuit or chip for sensing a voltage or capacitive change at a position on the screen which will then correlate with a command or icon at the same position, and allow for user input based on their contact with the screen.

In both instances, when a finger hits the screen at any position, a very small electrical charge is transferred to the finger of the user to complete the circuit. This transfer creates a voltage drop at pixel points on the screen which also host command keys or icons which the user is contacting to issue a command. Based on the position of the touch, and the command key or icon occupying the same position, and the relation of the command key or icon to a table of commands or actions, the software running the input of the touch screen processes the location of this voltage drop and orders the ensuing correlating action. Thus, users operating computer devices which require touch input to operate, continually contact their screens with their fingers which have been in contact with unknown pathogens.

Adding to the problem of a continual requirement for the user to touch or place a finger proximate the screen to operate the graphic interface software is the fact that the user in doing so, is continually depositing new and varied pathogens during their use of the touch screen. For example, users traveling through airports and on planes, in touching surfaces in such places that abound with pathogenic occupants, acquire new pathogenic occupants who take up residence on their touch screens when the user subsequently touches the screen during use. Thus, the population of such pathogens not only survives on such touch screens for long periods, when the device is used by the user, new populations of pathogens are continually deposited on such computers, smart phones, tablets, and other devices operated by a user which employ a touch screen interface to operate. Screen cleaners currently exist, however, conventionally such devices generally employ liquid sprays or wipes or provide a moist towelette which is impregnated with a liquid disinfectant such as alcohol or other liquid disinfectant cleaners. These devices were originally marketed for removing smudges and dirt from the glass or plastic surface of touch screens for which rubbing alcohol worked well as it is a well-known cleaner for windows. More recently, such glass cleaners have been marketed for disinfection and some have even included antibiotic or bacterial components.

However, almost all electronic device manufacturers recommend against placing a liquid anywhere proximate to an electronic device. Consequently, the use of wet towelettes is not a recommended course of action for any type of contact with the delicate electronics of modern smartphones and pad computers. Further, while a wipe with an alcohol-soaked towelette may clean off the dirt and smudges and remove some of the pathogens, once the alcohol evaporates there remains no long term deterrent to populations of new pathogenic occupants. Still further, the use of alcohol and many disinfectant liquids which may contain it or peroxide or the like, will also strip the fingerprint and smudge-resistant oleophobic coating from the screen.

Additionally, as noted, bacteria have been employing charge shifts to fend off modern antibacterial removal and the addition of a charged surface of the touch screen could enhance that ability for bacteria and pathogens to attempt to form a protective charged cover.

As such, there is an unmet need for a simple and portable means for disinfecting computers, smartphones, and other touch surfaces which are placed in contact with a single user, or multiple users. Such a device should be configured to kill resident pathogens such as germs and bacteria and viruses which populate touch surfaces on contact. Such a device should additionally impart a long term pathogen deterrent to the touch surface which will remain active after the initial removal. Further, such a device and method should be simple to employ, compact, and provide users with continually renewable protection against a return of a pathogen population on a touch surface interface such as smartphone screens. Finally, such a device and method should immediately remove and continually deter bacteria and the like including those which have a charged cover, through the employment of charged particles which will destroy even charged skinned pathogens.

The forgoing examples of related art and limitations related therewith are intended to be illustrative and not exclusive, and they do not imply any limitations on the pathogen removal and prevention invention described and claimed herein. Various additional limitations of the related art will become apparent to those skilled in the art upon a reading and understanding of the specification below and the accompanying drawings.

### Objects of the Invention

It is an object of the present invention to provide a device and method which allows users of portable computers, smartphones, and other electronic devices employing a touch screen interface for operation, to disinfect the touch screen of resident pathogens such as bacteria, viruses and germs.

It is an additional object of this invention to provide such a device and method which is compact, and employable on multiple successive occasions to provide a long term solution to hand and finger contact with touch screen interfaces having pathogen populations.

It is a further object of this invention, to provide such a pathogen deterrent and removal system and device, which on use not only removes current pathogen residents, but also imparts a residual deterrent to the screen or surface to deter pathogen populations until the next use of the device.

It is yet another object of this invention to provide a deposited pathogen deterrent and removal material to the touch screen which is configured to remove even pathogens which have formed a charged cover which renders other antibiotic materials useless.

Yet another object of this invention is the provision of such a pathogen deterrent and removal device and method, which employs dry material rather than a liquid to thereby prevent damage to electronic devices caused by liquid contact.

These and other objects, features, and advantages of the present invention, as well as the advantages thereof over existing prior art, which will become apparent from the description to follow, are accomplished by the novel improvements described in this specification and hereinafter of the touch screen interface pathogen removal and deterrent as described in the following detailed description which fully discloses the invention, which however in no manner should be considered as placing any limitations thereon.

### SUMMARY OF THE INVENTION

In accordance with the purposes of the present invention, as embodied and broadly described herein, the present invention provides a method and apparatus for sanitizing a touch surface screen used for computer operation and interface, as well as imparting material to the screen which will kill pathogenic occupants immediately and for a subsequent period after a sanitizing.

In operation for sanitizing and disinfecting a touch screen interface, in a particularly preferred mode, a microfiber cloth is employed which is infused with highly ionic metal oxide particles. The microfiber cloth is placed in a sandwiched contact between the finger of the user on one side, and the touch screen being cleaned on the other. In use as the user presses and rubs the microfiber cloth in contact against the screen to clean it.

During this contact some of the nanoparticles of metal ions or metal are imparted to the microfiber cloth, and some are communicated against and transferred to the touch screen.

As noted, in using the touch screen, when a finger touches a touch screen of a display of a touch computer such as a smartphone for example, the electric field at the nearest intersecting wires in or adjacent the glass in the proximity to the touch, grows so more electrical charge is stored on the glass surface. This de-powers the wires in that X-Y sector touched, and a sensor monitoring current on the screen surface discerns that while all the other wires of the grid are producing the standard amount of current, the area of the screen where the wires that intersect near a touching finger, are communicating the presence of a different current level.

As the electrostatic field of the touch screen changes at the point of finger contact, and depending on the situation, the field will change to be slightly more negative or positive.

During the act of use of the device and the compression and rubbing of the microfiber cloth against the screen surface by the user's finger, the user's finger is placed sufficiently proximate to the screen, to cause such an electrical field change at the surface to negative or positive, depending on the situation. At this point, because the metal nanoparticles deposited to the cloth have both positive and negatively charged metal ion particles, depending on the electrical field produced on the screen surface at the contact point with the microfiber cloth, opposite the user's finger pressing the cloth, either a positively or negatively charged metal nanoparticle is attracted to the opposite charged area to thereby temporarily engaged a deposit metal material or metallic ions on the touch screen surface. These nanoparticles preferably in metal ions attracted by the respective opposite charge, remain on the screen surface being held there by the charge force.

In this action, a finger placed proximate to the screen and causing a positively charged area on the grid will cause the attraction from the microfiber cloth of a negatively charged nanoparticle of metal or ionized metal to the screen surface proximate to that positive charge. Conversely, a touch to the screen with the cloth by a pressing of a finger which forms a negatively charged area on the surface, will result in the attraction and deposit of ionized metal nanoparticles which have a positive charge to the screen.

With both negatively and positively charged particles positioned within the cloth, which are then deposited on the screen in this fashion by a biased rubbing of the microfiber cloth against the touch screen, the deposited negatively charged nanoparticles of metal or metal ions will seek and kill a positively charged germ which comes into contact. Conversely, the negatively charged area on the grid which attracts and holds the metal nanoparticles of metal or metal ions that carry a positive charge, will subsequently be attracted and attach to a negatively charged pathogen.

Thus, because nanoparticle metal or metal ions with both positive and negative charges are imparted to the cloth, contact of those metal particles with pathogens during the rubbing of the cloth will immediately kill contacted pathogens. Metal nanoparticles or ionic metal particles which do not make engagement with a pathogen, will remain held to the screen surface by the charge attracting them and will continue to kill subsequent pathogens calling the screen surface home, afterward.

The negatively charged and positively charged metal nanoparticles or ionic particles which are imparted to the microfiber cloth, during manufacture and during any resupply, are communicated to the cloth using a pressurized stream of ultra-fine or nanoparticle sized, highly ionic metal oxide nanoparticles, or by tumbling the cloth in a closed container housing a supply of such nanoparticles.

The metal nanoparticles herein employed as noted, are nanoparticle-sized metal preferably ionic metal, which as used herein, are microscopic particles with at least one dimension less than 150 nm. Thus, the nanoparticle sized metallic material or ionic metallic material herein employs nanoparticle material having at least one dimension between 1 and 150 nm. These nanoparticles employed are included from a group of metal particles and include one or a combination of ionic particle types, from a group of metallic nanoparticles including zinc oxide, silver, titanium oxide, brass, copper, aluminum, and other metal ions.

Once the powdered mixture of nanoparticle metal or ionic metal material is operatively engaged to the cloth, they will remain trapped in-between the small fibers forming the yarn or thread of which the cloth is woven or knitted. So positioned, the particles will remain frictionally engaged to the fibers until sufficient electrical attraction draws them to be deposited to the touch screen during a sanitizing as noted depending on the electric charge.

As noted, when these metal nanoparticles or ionic particles come in contact with a pathogen, such as bacteria or a germ, they cause the outer surface of the pathogen to oxidize and yields a resulting break down of the barrier properties of that surface. Once this cell surface is compromised, it becomes porous and the nanoparticles of metal or metal ions move to a contact with the interior of the pathogenic cell. This contact is equally harmful to the cell as it causes a cessation of the cell's ability to utilize energy and stops cell DNA from replicating, and can impair the enzymes used to make oxygen.

These actions by the particles deposited by electrical attraction from the saturated cloth, to the touch screen, not only kill the pathogens but additionally those pathogens of the same ilk are rendered unable to develop immunity to the nanoparticle metal or metal ionized material. This is because during the time they might be starting to develop such immunity, they are being attacked with such swiftness by the piercing of their outer layer, that they expire in short order and before they can start to adapt.

One preferred metallic ion nanoparticle for the device herein is the silver ionic nanoparticle. Such silver ions are configured in a manner that they have more exterior surface area than many other metallic particles. This larger surface area is employed to more quickly and better surround a pathogen such as a bacteria or germ, and additionally increases the area placed in contact with the pathogen which increases its effectiveness. Experimentation with silver ion particles imparted to the cloth of the device herein has shown significantly enhanced pathogen eradication and prevention. Silver nanoparticles or ion particles employed averaged between 8.5 nanometers to as small as 1.9 nm.

However, it was found that by imparting silver nanoparticles or silver ion particles between 4.5 nm and 5.7 nm to the microfiber cloth, that the pathogen eradication was enhanced as was the long term eradication to pathogens later encountering the touch screen. As such, imparting such nanoparticles of silver or ionic silver in a size range between 4.5 and 5.7 nm with both positive and negative charges in the mix, is a particularly preferred mode of populating the cloth with nanoparticle metal or ionic metal material.

Another particularly preferred metallic nanoparticle or ion metal material used is zinc oxide. In experimentation, as noted above, while the silver metal nanoparticles or metal ions noted above had an excellent eradication and protection ability on touch screens, a downside was discovered that ionic or nanoparticle metallic silver material can tend to turn surfaces and skin a bluish color. While this change of color is harmless, further experimentation has shown that nanoparticle zinc oxide imparted to the microfiber cloth, was equally adept at removing and preventing subsequent pathogens from occupying surfaces such as touch screens but did not suffer from a color change issue.

Consequently, zinc oxide or titanium oxide nanoparticles are another particular favorite of the invention herein when imparted to the microfiber cloth used to clean and disinfect a touch screen or surface. Alternatively, a mixture of primarily zinc oxide nanoparticles or titanium oxide particles along with a small amount of silver particles such as a 90 to 10 ratio of zinc oxide or titanium oxide nanoparticles to silver nanoparticles, has been shown to allow the silver particles to work in concert with the zinc oxide or titanium oxide, but minimizes or eliminates to bluish colorizing. By adding the silver nanoparticles in such a small amount, any pathogen which might be resistant to the nanoparticle zinc oxide is eradicated by the silver nanoparticles and vice versa and a preferred outcome. With respect to the above description, before explaining at least one preferred embodiment of the herein disclosed touch screen interface pathogen removal and deterrent in detail, it is to be understood that the invention is not limited in its application to the details of construction and to the arrangement of the components in the following description or illustrated in the drawings. The device herein described and disclosed in the various modes and combinations is also capable of other embodiments and of being practiced and carried out in various ways which will be obvious to those skilled in the art. Any such alternative configuration as would occur to those skilled in the art is considered within the scope of this patent. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other pathogen removal and deterrent devices for sanitizing and disinfecting touch interfaces and for carrying out the several purposes of the present disclosed device. It is important, therefore, that the claims be regarded as including such equivalent construction and methodology insofar as they do not depart from the spirit and scope of the present invention.

### BRIEF DESCRIPTION OF DRAWING FIGURES

The accompanying drawings, which are incorporated herein and form a part of the specification, illustrate some, but not the only nor exclusive examples of embodiments and/or components of the disclosed device. It is intended that the embodiments and figures disclosed herein are to be considered illustrative of the invention herein, rather than limiting in any fashion.

In the drawings:

FIG. 1 depicts a touch screen device having a grid of wires which provide signals for computer action when the screen is touched.

FIG. 2 depicts the device herein being employed to disinfect and to provide long term pathogen detergence to the screen of FIG. 1.

FIG. 3 depicts a cotton or similar fiber cloth having a much lower area of space for occupancy of positive and negatively charged metal nanoparticles or ionic metal nano particles.

FIG. 4 shows the preferred mode of the device showing a strand of microfiber yarn with a very high area and affinity for negative and positive metal nanoparticles or metal ions for deposit on the opposite electric charge on a touch screen mixing with pathogens on the screen and in the passages of the yarn.

FIG. 5 depicts one mode of provision of the device housed in a foil or plastic package with a microfiber component imparted with a supply of metal nanoparticles.

FIG. 6 shows a sliced view through a resealable mode of the package of FIG. 5 showing the folded material with imparted metallic nanoparticles or metallic ion particles which are also positioned within the package for resupply.

### DETAILED DESCRIPTION OF THE INVENTION

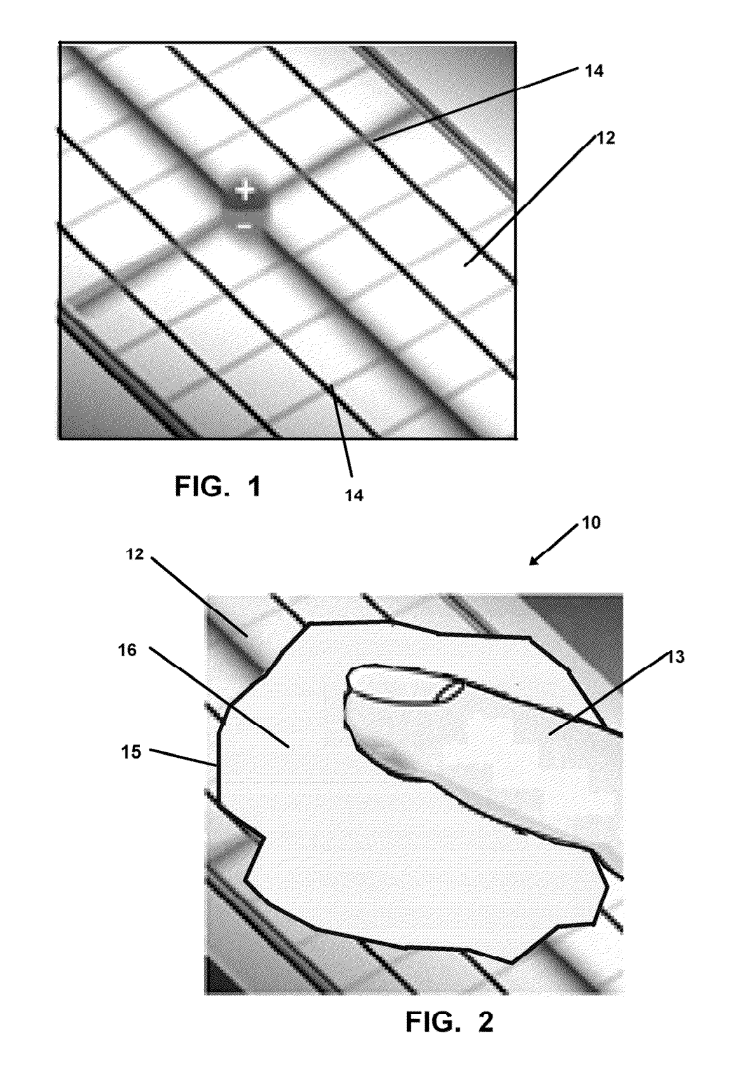

Now referring to drawings in FIGS. 1-6, wherein similar components are identified by like reference numerals, there is seen in FIG. 1 a glass touch screen 12 having a grid of wires 14 which are operatively positioned to provide electronic signals recognized by software adapted to the task. The software acting on the determined location of the electronic signal generated on the grid of wires 14, initiates a related action. As noted, the touch screen 12 tends to become soiled and smudged and also harbors pathogens such as bacteria, viruses, and other infectious life forms.

As shown in FIG. 2, the device 10 and system herein employs preferably a microfiber cloth 16 to which nanoparticle metal or metal ion material has been imparted. A flexible applicator 15, formed of cloth or preferably microfiber material 16 is formed in a woven or non woven fabric of very fine fibers compared to more conventional fabrics using larger denier yarn. As used herein, to provide a measure for comparison, microfibers employed in forming such cloth, are half the diameter of a fine silk fiber which averages 15 micrometers, and one-third the diameter of cotton which averages 21 micrometers in diameter. Thus, the denier of a filament of microfiber material 16, is significantly smaller than that of conventional materials such as cotton or silk or man-made fibers.

Denier is the term used to define the diameter or fineness of a continuous or twisted filament fiber such as silk or man-made fibers. Denier is the weight in grams of a 9000-meter length of fiber or yarn and thus the higher the number, the thicker the fiber. In general, in order to be termed a “microfiber,” the fiber must be less than one denier. In comparison, fine silk, for example, is approximately 1.25 denier. Thus, a microfiber is conventionally to be 0.9 denier or finer. A large majority of microfibers manufactured are from 0.5 to 0.9 denier.

In another example, very fine nylon stockings are knit from 10 to 15 denier yarns, with each yarn consisting of 3 to 4 filaments which are twisted to form the yarns. A 15 denier yarn made of microfiber can have as many as 30 filaments which are twisted to form the yarn. It is this high number of filaments twisted to form the very fine yarn which also yields a much more extensive surface area determined by the surfaces of the many filaments and the spaces between these individual filaments.

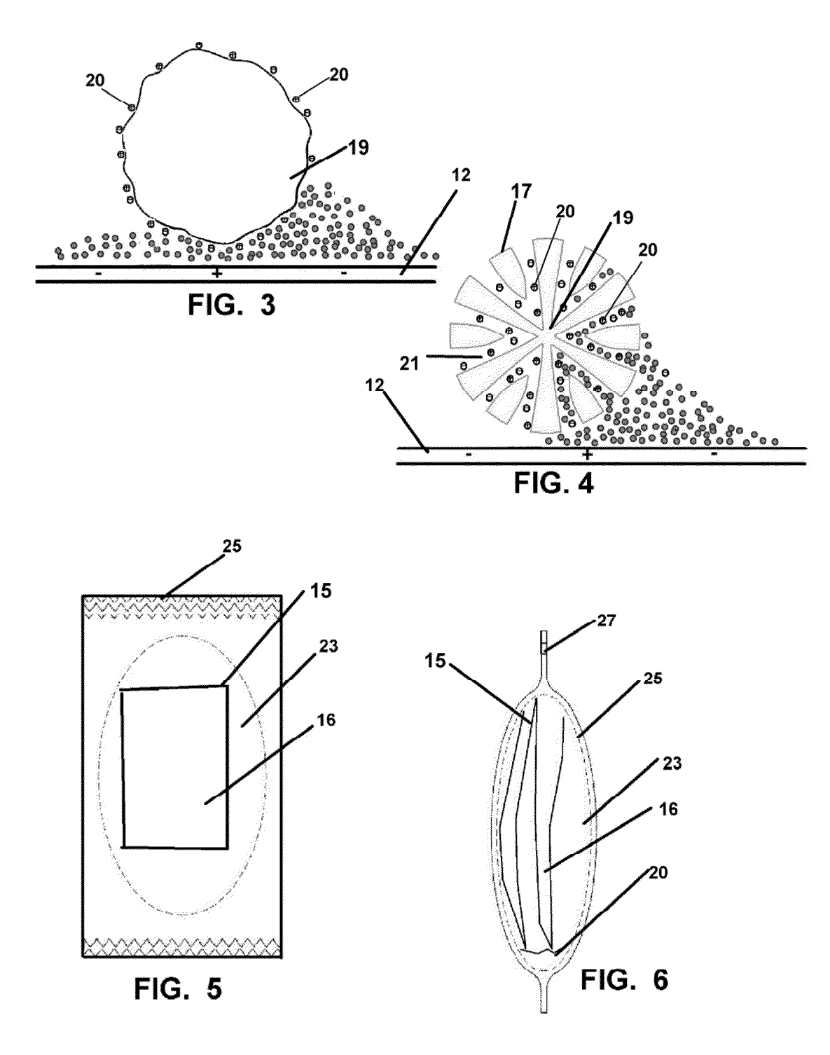

Experimentation has found that as shown in FIG. 3, conventional fibers of twisted filaments have much less area on the perimeter for charged metallic nanoparticles or ionic metal nanoparticles to occupy. However, it was found that the very small gaps descending into microfiber yarn forming the microfiber material 16 (FIG. 1), which render it soft to the touch, also provide an exceptionably large surface area compared to yarns forming other fabric material such as cotton as shown in FIG. 3.

It is in this narrow but plentiful surface area, between filaments or descending into the filaments forming the microfiber yarns formed to the microfiber material 16 (FIG. 1), where metal nanoparticles 20 are imparted and stay frictionally engaged until dislodged or until they encounter a pathogen. Further, in these narrow passages depending into the yarn forming the microfiber material, pathogens are drawn and can mix and be dispatched in an encounter with the nanoparticles such as in FIG. 4.

As can be discerned, the surface area and descending areas of each of 30 filaments forming a 15 denier yarn of microfiber such as in FIG. 4, significantly exceeds the surface area of each of 3 to 4 filaments twisted to form nylon stocking yarn or cotton yarn such as in FIG. 3. Further the spaces between the twisted filaments of the microfiber yarn form gaps descending therein which are particularly well sized and suited to frictionally engage metal nano particles therein. As such the employment of microfiber material 16 (FIG. 1), formed of yarn 0.9 denier or less, is particularly preferred in that it significantly increases the amount of metal nanoparticles which may be imparted to the microfiber cloth used herein.

As shown in FIG. 4, the imparted metal nano particles 20 may be sprayed, tumbled, dropped, or otherwise imparted to the microfiber material 16 (FIG. 1), and will immediately engage to the filament surfaces in the very small spaces 21, between the multiple filaments 17 formed to the microfiber yarn 19 strands.

The nanoparticle sized metal particles or metal ions shown as nanoparticles 20 herein is formed of metal nanoparticles which have at least one dimension between 1 and 150 nm. Preferably sized in this fashion, the nanoparticles 20 herein are metal nanoparticles or metal ions including one or a combination of metal nanoparticles, from a group of metal nanoparticles of similar or ionic configuration, including zinc oxide, titanium oxide, silver, brass, copper, aluminum.

A particular favorite being titanium oxide and/or zinc oxide nanoparticles 20 solely, or in a combination with silver nanoparticles 20, in a ratio of between 80 to 99 percent zinc oxide or titanium oxide, to 1 to 20 percent silver particles, of the total mixture. One particular favorite is a mixture of 90 percent zinc oxide and/or titanium oxide nanoparticles and 10 percent silver in nanoparticles which as noted above worked exceptionally well to eliminate and prevent re-occupancy of pathogens on touch screens.

In operation for sanitizing and disinfecting a touch screen 12 as seen in FIGS. 2 and 4, the microfiber material 16 which is infused with highly metal oxide nanoparticles 20 which are preferably ionic in that they have different nanoparticles with opposite charges, which occupy the significant amount of space depending into the formed yarn, is swiped or rubbed upon the touch screen 12. As shown in FIG. 2, the microfiber material 16, is placed in a sandwiched contact between the finger 13 and the touch screen 12 being cleaned.

In this fashion, as the user presses and rubs the microfiber material 16 in contact against the touch screen 12 to clean it, some of the metal nanoparticles 20 imparted to the microfiber material 16, are communicated against, and transferred to, the surface of the touch screen 12 such as in FIG. 4.

The electrostatic field of the touch screen 12 changes at the point of finger and microfiber cloth 16 contact, and depending on conditions the field will change to be slightly more negative or positive. Consequently, nanoparticles 20 with the opposite charge as depicted in FIG. 3 will be attracted to and attach to touch screen 12 areas of the opposite charge as in FIG. 4. Because of the high volume of surface area provided by the microfiber material 16 as noted above, there is an ample supply of nanoparticles 20 of both charge to occupy the touch screen 12. So positioned, these nanoparticles 20 formed of one or mixture of metal nanoparticles preferably ionic in nature to provide both charges, will remove pathogens of both charges and inhibit their return to the touch screen 12.

As shown in FIG. 5, the device 10 can provide the nanoparticle infused microfiber material 16 housed in a sealed cavity 23 of a foil or plastic package 25. In this mode of FIG. 5, the microfiber material 16 infused with nanoparticles 20 can be removed from the torn package 25, used once, and discarded.

In FIG. 6 is shown a resealable mode of the package 25 of FIG. 5 where a zip lock 27 or other resealable opening is provided. In this mode the microfiber material 16 imparted with metallic nanoparticles 20 preferably ionic in charge of the included particles, and may be replenished be placement back in the package 25 and shaking it to cause the supply of nanoparticles 20 to impart to the yarn forming the microfiber material 16.

As noted, any of the different configurations and components can be employed with any other configuration or component shown and described herein to form the device or employ the method. Additionally, while the present invention has been described herein with reference to particular embodiments thereof and steps in the method of production, a latitude of modifications, various changes and substitutions are intended in the foregoing disclosures, it will be appreciated that in some instance some components, or configurations, or steps in formation and/or use of the invention could be employed without a corresponding use of other components without departing from the scope of the invention as set forth in the following claims. All such changes, alternations and modifications as would occur to those skilled in the art are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. A pathogen removal and deterrent apparatus for touch screens employed on user input devices, comprising:
a flexible applicator sized for a sandwiched employment between a part of a user's hand, and said touch screen in a biased contact of said applicator, with said touch screen;
said applicator formed of yarn; and
said yarn infused with metallic nanoparticles having an affinity for a communication with pathogens in position on said touch screen, whereby said communication of a said metallic nanoparticle with a said pathogen, causing an eradication of said pathogen.

2. The pathogen removal and deterrent apparatus of claim 1, additionally comprising:
said yarn formed of filaments forming microfiber yarn;
said microfiber yarn having gaps between said filaments exposed to said touch screen during said contact therewith;
said gaps sized to frictionally maintain a reservoir of said metallic nanoparticles therein, prior to said contact; and
said contact causing a deposit of said metallic nanoparticles to said touch screen, from said gaps, and concurrently a drawing of said pathogens into said gaps.

3. The pathogen removal and deterrent apparatus of claim 1 wherein said metal nanoparticles infused to said yarn, include one or a combination of nanoparticles from a group consisting of zinc oxide, titanium oxide, silver, brass, copper, and aluminum.

4. The pathogen removal and deterrent apparatus of claim 2 wherein said metal nanoparticles infused to said yarn, include one or a combination of nanoparticles from a group consisting of zinc oxide, titanium oxide, silver, brass, copper, and aluminum.

5. The pathogen removal and deterrent apparatus of claim 3 wherein said nanoparticles have at least one dimension between 1 and 150 nm.

6. The pathogen removal and deterrent apparatus of claim 4 wherein said nanoparticles have at least one dimension between 1 and 150 nm.

7. The pathogen removal and deterrent apparatus of claim 5 wherein said nanoparticles are ionic metal nanoparticles in a mixture having said nanoparticles of both positive and negative charges.

8. The pathogen removal and deterrent apparatus of claim 6 wherein said nanoparticles are ionic metal nanoparticles in a mixture having said nanoparticles of both positive and negative charges.

9. The pathogen removal and deterrent apparatus of claim 3 wherein said nanoparticles infused to said yarn, are a mix of said nanoparticles in a total mixture and are mixed in a ratio of between 80 to 99 percent zinc oxide or titanium oxide, to 1 to 20 percent silver particles, of the total mixture.

10. The pathogen removal and deterrent apparatus of claim 4 wherein said nanoparticles infused to said yarn, are a mix of said nanoparticles in a total mixture and are mixed in a ratio of between 80 to 99 percent zinc oxide or titanium oxide, to, 1 to 20 percent silver particles.

11. The pathogen removal and deterrent apparatus of claim 5 wherein said nanoparticles infused to said yarn, are a mix of said nano particles in a total mixture and are mixed in a ratio of between 80 to 99 percent zinc oxide or titanium oxide, to, 1 to 20 percent silver particles.

12. The pathogen removal and deterrent apparatus of claim 6 wherein said nanoparticles infused to said yarn, are a mix of said nano particles in a total mixture and are mixed in a ratio of between 80 to 99 percent zinc oxide or titanium oxide, to, 1 to 20 percent silver particles.

13. The pathogen removal and deterrent apparatus of claim 7 wherein said nanoparticles infused to said yarn, are a mix of said nanoparticles in a total mixture and are mixed in a ratio of between 80 to 99 percent zinc oxide or titanium oxide, to, 1 to 20 percent silver particles.

14. The pathogen removal and deterrent apparatus of claim 8 wherein said nanoparticles infused to said yarn, are a mix of said nanoparticles in a total mixture and are mixed in a ratio of between 80 to 99 percent zinc oxide or titanium oxide, to, 1 to 20 percent silver particles.

15. The pathogen removal and deterrent apparatus of claim 3 wherein said nanoparticles infused to said yarn, are a mix of said nano particles in a total mixture are mixed in a ratio of 90 percent zinc oxide and/or titanium oxide nanoparticles and 10 percent silver.

16. The pathogen removal and deterrent apparatus of claim 4 wherein said nanoparticles infused to said yarn, are a mix of said nanoparticles in a total mixture and are mixed in a ratio of 90 percent zinc oxide and/or titanium oxide nanoparticles and 10 percent silver.

17. The pathogen removal and deterrent apparatus of claim 5 wherein said nanoparticles infused to said yarn, are a mix of said nanoparticles in a total mixture and are mixed in a ratio of 90 percent zinc oxide and/or titanium oxide nanoparticles and 10 percent silver.

18. The pathogen removal and deterrent apparatus of claim 6 wherein said nanoparticles infused to said yarn, are a mix of said nanoparticles in a total mixture and are mixed in a ratio of 90 percent zinc oxide and/or titanium oxide nanoparticles and 10 percent silver.

19. The pathogen removal and deterrent apparatus of claim 7 wherein said nanoparticles infused to said yarn, are a mix of said nanoparticles in a total mixture and are mixed in a ratio of 90 percent zinc oxide and/or titanium oxide nanoparticles and 10 percent silver.

20. The pathogen removal and deterrent apparatus of claim 8 wherein said nanoparticles infused to said yarn, are a mix of said nanoparticles in a total mixture and are mixed in a ratio of 90 percent zinc oxide and/or titanium oxide nanoparticles and 10 percent silver.
